(19) United States
(12) Patent Application Publication
(10) Pub. No.: US 2023/0255694 A1
(43) Pub. Date: Aug. 17, 2023

(54) SYSTEMS AND METHODS FOR VALIDATING A POSE OF A MARKER

(71) Applicant: Mazor Robotics Ltd., Caesarea (IL)

(72) Inventors: Ziv Seemann, Beit Ytzhack (IL); Dvir Kadshai, Tel Aviv (IL); Itamar Eshel, Tzur Igal (IL); Yuval A. Chen, Tel Aviv-Yafo (IL); Nimrod Dori, Atlit (IL); Nicholas J. Rawluk, Boulder, CO (US)

(21) Appl. No.: 17/671,893

(22) Filed: Feb. 15, 2022

(51) Int. Cl. A61B 34/20; A61B 34/30

(52) U.S. Cl. CPC A61B 34/20; A61B 34/30; A61B 2034/2068; A61B 2034/2057

(57) ABSTRACT

Systems and methods for validating a pose of a marker are provided. First pose information and second pose information of the marker may be received. A pose difference between the first pose information and the second pose information may be determined. A pose of the marker may be validated in response to determining that the pose difference is less than a pose threshold.

### BACKGROUND

The present disclosure is generally directed to validation, and relates more particularly to validating a pose of a marker using an imaging device.

Surgical robots may assist a surgeon or other medical provider in carrying out a surgical procedure, or may complete one or more surgical procedures autonomously. A navigation system may be used by a medical provider for tracking and/or providing navigation of one or more components. The navigation system may use a camera and/or a sensor for tracking the one or more components.

### BRIEF SUMMARY

Example aspects of the present disclosure include:

A system for validating a pose of a marker according to at least one embodiment of the present disclosure comprises a marker; a processor; and a memory storing data for processing by the processor, the data, when processed, causes the processor to: receive first pose information of the marker; receive second pose information of the marker; determine a pose difference between the first pose information and the second pose information; and validate a pose of the marker in response to determining that the pose difference is less than a pose threshold.

Any of the aspects herein, further comprising a first imaging device and a second imaging device, wherein the first pose information is received from the first imaging device and the second pose information is received from the second imaging device.

Any of the aspects herein, wherein the first imaging device comprises a navigation camera and the second imaging device comprises a three-dimensional camera.

Any of the aspects herein, wherein the second imaging device is positioned on a robot and the marker is positioned on a patient.

Any of the aspects herein, wherein the second imaging device is positioned external to a robot and the marker is positioned on the robot.

Any of the aspects herein, wherein the memory stores further data for processing by the processor that, when processed, causes the processor to: calculate an accuracy index based on the pose difference; and display the accuracy index.

Any of the aspects herein, wherein the memory stores further data for processing by the processor that, when processed, causes the processor to: monitor the pose difference; and automatically generate a notification when the pose difference exceeds the pose threshold.

A system for validating a pose of a marker according to at least one embodiment of the present disclosure comprises a navigation system comprising a first imaging device; a second imaging device positioned on a robot; a processor; and a memory storing data for processing by the processor, the data, when processed, causes the processor to: receive first pose information from the first imaging device; receive second pose information from the second imaging device; determine a pose difference between the first pose information and the second pose information; and validate a pose of the marker in response to determining that the pose difference is less than a pose threshold.

Any of the aspects herein, further comprising a marker.

Any of the aspects herein, wherein the marker is positioned on an instrument.

Any of the aspects herein, wherein the marker comprises at least one light emitting diode.

Any of the aspects herein, wherein the instrument comprises a bone mount.

Any of the aspects herein, wherein the first pose information corresponds to a pose of the marker and the second pose information corresponds to a pose of the instrument.

Any of the aspects herein, wherein the first imaging device is a navigation camera and the second imaging device is a three-dimensional camera.

A system for validating a pose of a marker according to at least one embodiment of the present disclosure comprises a marker positioned on a robot; a navigation system comprising a first imaging device; a second imaging device positioned external to the robot; a processor; and a memory storing data for processing by the processor, the data, when processed, causes the processor to: receive a first pose information of the marker from the first imaging device; calculate a first pose of the marker in a robot coordinate space based on the first pose information; receive a second pose information of the marker from the second imaging device; calculate a second pose of the marker in the robot coordinate space based on the second pose information; determine a pose difference between the first pose and the second pose; and validate a pose of the marker in response to determining that the pose difference is less than a pose threshold.

Any of the aspects herein, wherein calculating the first pose of the marker in the robot coordinate space is further based on first pose information of a robot reference point and calculating the second pose of the marker in the robot coordinate space is further based on second pose information of the robot reference point.

Any of the aspects herein, wherein the robot reference point comprises at least one of a robot reference frame or a robot landmark.

Any of the aspects herein, wherein the first imaging device is a navigation camera and the second imaging device is a three-dimensional camera.

Any of the aspects herein, wherein the marker comprises at least one of a light emitting diode, an infrared light emitting diode, or a reflective sphere.

Any of the aspects herein, wherein the memory stores further data for processing by the processor that, when processed, causes the processor to: monitor the pose difference; and automatically generate a notification when the pose difference exceeds the pose threshold.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2 is a flowchart according to at least one embodiment of the present disclosure; and

FIG. 3 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

During a surgical operation, a navigation system may be used to track one or more components during the surgical operation. The navigation system includes a navigation camera to track the one or more components. If a navigation camera is not working properly, conventional methods for troubleshooting the navigation camera includes using anatomical landmarks or reference tracker divots. However, anatomical landmarks may not be accurate and does not have actual measurements and using reference tracker divots is a manually induced process and no indication other than a surgeon's judgement is used to initiate the process.

According to at least one embodiment of the present disclose, a three-dimensional camera (or any type of camera) may be used to see and identify a marker versus a robot reference point to know the marker's location in “robot space.” Then, the same marker and robot reference frame is seen and identified by the navigation camera, so the marker's location in “robot space” is known. The navigation accuracy can be verified by using the data from the three-dimensional camera and the navigation camera at the same time. The error between the two readings may be evaluated and if the error is greater than an error threshold, then a user (such as, for example, a surgeon or other medical provider) may be notified. The three-dimensional camera can be on the robot, near the navigation camera, or a handheld device. If the three-dimensional camera is on the robot it can be calibrated and only needs to see a tool geometry. If the camera is not on the robot, it needs to be able to see the navigation reference tracker or a landmark on the robot (e.g., an arm guide) and then calculate a robotic position and to connect it to the anatomy.

Using this embodiment, the navigation camera accuracy can be quantified and a limit for troubleshooting the navigation camera can be set. A “live” navigation accuracy index may be shown to a user such as the surgeon or other medical provide. Such live verification may prevent a non-accurate system from working and may reduce risk of injury or a bad implant placement due to an inaccurate system. The process may be done automatically without a user initiating the action.

In another embodiment of the present disclose, light emitting diodes (LEDs) may be incorporated in a tool so that the tool can be seen as a solid item in the three-dimensional camera and the LEDs can be seen by the navigation camera as markers. Both locations can be compared in real-time to confirm if the system is accurate.

Embodiments of the present disclosure provide technical solutions to one or more of the problems of (1) quantifying a navigation camera accuracy, (2) verifying an accuracy of a navigation camera, (3) providing real-time accuracy of a navigation camera, and (4) increasing safety of a patient and a surgical team.

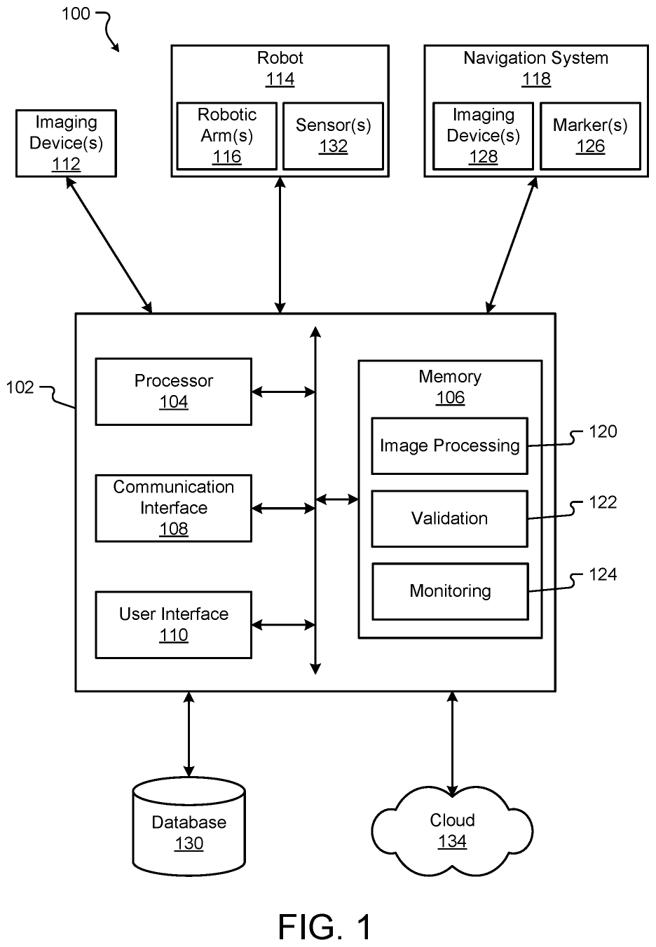

Turning first to FIG. 1, a block diagram of a system 100 according to at least one embodiment of the present disclosure is shown. The system 100 may be used to validate a pose of a marker such as a marker 126 and/or carry out one or more other aspects of one or more of the methods disclosed herein. The system 100 comprises a computing device 102, one or more imaging devices 112, a robot 114, a navigation system 118, a database 130, and/or a cloud or other network 134. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the methods 200 and/or 300 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120, validation 122, and/or monitoring 124.

The image processing 120 enables the processor 104 to process image data of an image (received from, for example, the imaging device 112, an imaging device 128 of the navigation system 118, or any imaging device) for the purpose of, for example, identifying information about at least one marker such as the marker 126 depicted in the image and/or identifying an object such as, for example, a tool, an instrument, a surgical landmark, etc. The information may comprise, for example, pose information of the marker 126 and/or pose information of the object. The pose information may correspond to computer-encoded data that describes a pose of the marker 126 and/or an object. For example, the pose information, in some embodiments, may comprise coordinates and/or an orientation of the marker and/or the object. In other examples, the pose information may comprise, for example, a matrix that describes the pose of the marker and/or the object. It will be appreciated that the pose information may be encoded in any number of ways and may include, for example, a description of a location of the marker and/or the object in a reference space, a vector (e.g., a three-element vector), or a matrix. The information such as the pose information obtained from the image processing 120 may enable the navigation system 118 to validate a pose of the marker 126.

The validation 122 enables the processor 104 (or a processor of the navigation system 118) to process the pose information (received from, for example, the image processing 120) for the purpose of, for example, validate a pose of the marker 126. The validation 122 may validate the pose of the marker 126 by comparing first pose information of the marker 126 received from a first imaging device (e.g., imaging device 128) and second pose information of the marker 126 received from a second imaging device (e.g., imaging device 112). More specifically, in some embodiments, the validation 122 may determine a pose difference between the first pose information and the second pose information. The validation 122 may then validate the pose of the marker 126 in response to determining that the pose difference is less than a pose threshold.

The monitoring 124 enables the processor 104 (or a processor of the navigation system 118) to monitor the marker 126 and more specifically, to monitor the pose difference. The monitoring 124 may, for example, enable the processor 104 to automatically validate the pose of the marker using the validation 122 by comparing the pose difference to the pose threshold. In other embodiments, the monitoring 124 may, for example, enable the processor 104 to automatically generate a notification if the pose difference is greater than the pose threshold. Such monitoring 124 provides for automatic and—in some embodiments—continuous monitoring of the pose difference and thus, automatic validation of the pose of the marker or generation of the notification if the pose difference is greater than the pose threshold.

The content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the robot 114, the database 130, and/or the cloud 134.

The computing device 102 may also comprise a communication interface 108. The communication interface 108 may be used for receiving image data or other information from an external source (such as the imaging device 112, the robot 114, the navigation system 118, the database 130, the cloud 134, and/or any other system or component not part of the system 100), and/or for transmitting instructions, images, or other information to an external system or device (e.g., another computing device 102, the imaging device 112, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a headset, keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or marker(s) such as markers(s) 126 to yield image data (e.g., image data depicting or corresponding to a bone, veins, tissue, etc. or the marker). “Image data” as used herein refers to the data generated or captured by an imaging device 112, including in a machine-readable form, a graphical/visual form, and in any other form. In various examples, the image data may comprise data corresponding to an anatomical feature of a patient or to a marker. The image data may be or comprise a preoperative image, an intraoperative image, a postoperative image, or an image taken independently of any surgical procedure. In some embodiments, a first imaging device, may be used to obtain first image data (e.g., a first image) at a first time, and a second imaging device 112 may be used to obtain second image data (e.g., a second image) at a second time after the first time. In other embodiments, a first imaging device may be used to obtain first image data and a second imaging device may be used to obtain second image data at substantially the same time. In at least some embodiments, the first imaging device may use a first imaging modality and the second imaging device may use a second imaging modality.

The imaging device 112 may be capable of taking a 2D image or a 3D image to yield the image data. The imaging device 112 may be or comprise, for example, a three-dimensional camera, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (Mill) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated. The imaging device 112 may be positioned on, for example, the robot 114, or may be positioned external to the robot 114.

The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces.

The robot 114, together with the robotic arm 116, may have, for example, one, two, three, four, five, six, seven, or more degrees of freedom. Further, the robotic arm 116 may be positioned or positionable in any pose, plane, and/or focal point. The pose includes a position and an orientation. As a result, an imaging device 112, surgical tool, or other object held by the robot 114 (or, more specifically, by the robotic arm 116) may be precisely positionable in one or more needed and specific positions and orientations.

The robot 114 may comprise one or more sensors 132. The sensor 132 may be a position sensor, a proximity sensor, a magnetometer, or an accelerometer. In some embodiments, the sensor 132 may be a linear encoder, a rotary encoder, or an incremental encoder. Other types of sensors may also be used as the sensor 132. The one or more sensors 132 may be positioned, for example, on the robotic arm 116 or elsewhere. Data from the sensor(s) 132 may be provided to a processor of the robot 114, to the processor 104 of the computing device 102, and/or to the navigation system 118. The data may be used to calculate a position in space of the robotic arm 116 relative to one or more coordinate systems. The calculation may be based not just on data received from the sensor(s) 132, but also on data or information (such as, for example, physical dimensions) about, for example, the robot 114 or a portion thereof, which data or information may be stored, for example, in a memory 106 of a computing device 102 or in any other memory.

In some embodiments, reference markers 126 (e.g., navigation markers) may be placed on the robot 114 (including, e.g., on the robotic arm 116), the imaging device 112, or any object or component in the surgical space. The marker 126 may comprise one or more active markers, one or more passive markers, or a combination of active and passive markers. The marker 126 may comprise, for example, light emitting diodes, infrared light emitting diodes, reflective markers, or the like. The marker 126 may be tracked by the navigation system 118 and the results of the tracking may be used by the robot 114 and/or by an operator of the system 100 or any component thereof. The navigation system 118 may also be configured to obtain pose information describing a pose of the marker 126, which may be used to determine a correlating pose of the marker 126 or of an object to which the marker 126 is coupled to.

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more imaging devices 128 or other sensor(s) for tracking one or more reference markers, navigated trackers, or other objects within the operating room or other room in which some or all of the system 100 is located. In some embodiments, the navigation system 118 may comprise one or more electromagnetic sensors. The one or more imaging devices 128 may be the same as or similar to the imaging device 112. In some embodiments, the one or more imaging devices 128 may be optical cameras, infrared cameras, or other cameras.

In various embodiments, the navigation system 118 may be used to track a position and orientation (e.g., a pose) of the imaging device 112, the robot 114 and/or robotic arm 116, and/or one or more surgical tools (or, more particularly, to track a pose of a navigated tracker such as the marker 126 attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of any of the methods 200 and/or 300 described herein. The system 100 or similar systems may also be used for other purposes.

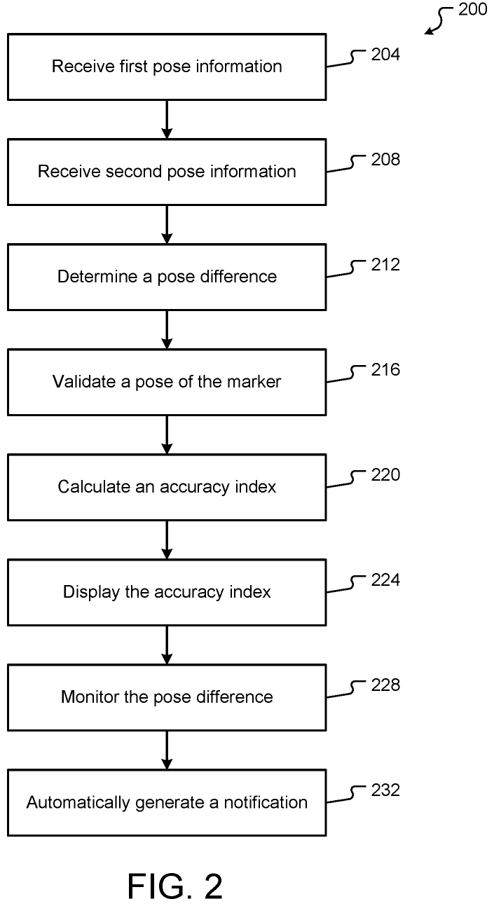

FIG. 2 depicts a method 200 that may be used, for example, for validating a pose of a marker.

The method 200 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 200. The at least one processor may perform the method 200 by executing elements stored in a memory such as the memory 106. The elements stored in the memory and executed by the processor may cause the processor to execute one or more steps of a function as shown in method 200. One or more portions of a method 200 may be performed by the processor executing any of the contents of memory, such as an image processing 120, validation 122, and/or monitoring 124.

The method 200 comprises receiving first pose information (step 204). The first pose information may be obtained from a first imaging device, which may be the same as or similar to the imaging device 128 of a navigation system such as the navigation system 118. In some embodiments, a processor such as the processor 104 may use image processing such as the image processing 120 to identify first pose information from image data received from the first imaging device.

In some embodiments, the first imaging device may comprise a navigation camera configured to track and obtain a pose of a marker such as the marker 126. The marker may comprise, for example, light emitting diodes, infrared light emitting diodes, reflective markers, or the like. In some embodiments, the marker may be positioned on a patient. In other embodiments, the marker may be positioned on the robot. In still other embodiments, the marker may be positioned on an instrument. The instrument may be, for example, a bone mount, a screw, or the like.

The method 200 also comprises receiving second pose information (step 208). The second pose information may be obtained from a second imaging device, which may be the same as or similar to the imaging device 112. In some embodiments, the processor may use the image processing to identify second pose information from image data received from the second imaging device.

In some embodiments, the second imaging device may comprise a three-dimensional camera configured to obtain a pose of an object or the marker. It will be appreciated that in some embodiments, the first imaging device and the second imaging device may use the same imaging modality. In other embodiments, the first imaging device may use a first imaging modality and the second imaging device may use a second imaging modality. The second imaging device may be positioned on a robot such as the robot 114. In other instances, the second imaging device may be positioned external to the robot. In other words, the second imaging device may not be positioned on the robot. The second imaging device may be positioned near, for example, the first imaging device.

In embodiments where the second imaging device is positioned on the robot and the marker is positioned on a landmark (e.g. an instrument), the first pose information may correspond to a pose of the marker and the second pose information may correspond to a pose of the landmark. In such embodiments, the first imaging device may comprise a navigational camera configured to obtain pose information of the marker and the second imaging device may comprise a three-dimensional camera configured to identify the landmark and obtain pose information of the landmark.

The method 200 also comprises determining a pose difference (step 212). Determining the pose difference may comprise a processor such as the processor 104 using a validation such as the validation 122 to determine the pose difference. The pose difference may comprise a difference between the first pose information (whether of a marker or an object) and the second pose information (whether of a marker or an object). In some embodiments, the pose difference is determined by subtracting one or more components of the first pose information from the corresponding components of the second pose information. The one or more components may comprise an x-coordinate, a y-coordinate, a z-coordinate, and/or an orientation (e.g., an angle of the marker or object). For example, the x-coordinate of the first pose information may be subtracted from the x-coordinate of the second pose information. Similarly, in other examples, a vector or a matrix of the first pose information may be subtracted from a corresponding vector or matrix of the second pose information.

The method 200 also comprises validating a pose of the marker (step 216). Validating the pose of the marker may comprise the processor using the validation 122 to determine that the pose difference obtained in, for example, step 212, is less than a pose threshold. The pose threshold may, for example, correspond to an allowable difference between the first pose information and the second pose information. The pose threshold may be determined automatically using artificial intelligence and training data (e.g., historical cases) in some embodiments. In other embodiments, the pose threshold may be or comprise, or be based on, surgeon input received via the user interface. In further embodiments, the pose threshold may be determined automatically using artificial intelligence, and may thereafter be reviewed and approved (or modified) by a surgeon or other user.

When the pose of the marker is validated, this indicates that the first imaging device (which may be, for example, the imaging device of the navigation system) is operating accurately. In other words, when the first pose information received from the first imaging device substantially matches the second pose information received from the second imaging device, this indicates that the first imaging device and the second imaging device are placing the marker in the same location. Thus, this also indicates that the first imaging device is accurate and functioning properly.

The method 200 also comprises calculating an accuracy index (step 220). Calculating the accuracy index may be based on the pose difference determined in step 212. The accuracy index may correlate to an accuracy of the first imaging device (which may be, for example, the imaging device of the navigation system) and/or an accuracy of the navigation system. In some embodiments, the accuracy index may comprise more than one accuracy index (e.g., an accuracy index for the first imaging device and an accuracy index for the navigation system). The accuracy index may be calculated continuously in real-time. In other instances, the accuracy index may be calculated at a time period. In still other instances, the accuracy index may be calculated upon input from a user or calculated at certain steps based on a surgical plan.

The method 200 also comprises displaying the accuracy index (step 224). The accuracy index may be displayed on, for example, a user interface such as the user interface 110. The accuracy index may be, for example, updated and displayed in real-time. In other embodiments, the accuracy index may be displayed upon input from a user. In still other embodiments, the accuracy index may be displayed as part of a notification, as described in step 232 below.

The method 200 also comprises monitoring the pose difference (step 228). Monitoring the pose difference may comprise the processor using a monitoring such as the monitoring 124 to monitor the marker and, more specifically, to monitor the pose difference determined in step 212. The monitoring may, for example, enable the processor to automatically validate the pose of the marker using the validation by comparing the pose difference to a pose threshold. In other embodiments, the monitoring may, for example, enable the processor to automatically generate a notification if the pose difference is greater than the pose threshold, as described below in step 232.

The method 200 also comprises automatically generating a notification (step 232). The notification may be a visual notification, an audible notification, or any type of notification communicated to a user. The notification may comprise, for example, the accuracy index, the pose difference, the first pose information, and/or the second pose information. The notification may be communicated to the user via a user interface such as the user interface 110. In some embodiments, the notification may be automatically generated by the processor using the monitoring. In other embodiments, the notification may be automatically generated by any component of a system such as the system 100. In some embodiments, the notification is based on the pose difference determined in step 212 and the pose threshold, as described in step 216. For example, the notification may be generated when the pose difference meets or exceeds the pose threshold. When the pose difference meets or exceeds the pose threshold, this may indicate that the first imaging device—which may be the imaging device of the navigation system—is currently inaccurate. In other words, when the first pose information received from the first imaging device does not substantially match the second pose information received from the second imaging device, this indicates that the first imaging device and the second imaging device are placing the marker in a different location in space. This may indicate that the first imaging device is not accurate and may not be functioning properly. As such, a notification to a user such as a surgeon or other medical provider can alert the user to such inaccuracy.

It will be appreciated that any of the steps may be continuously repeated to provide real-time validation and monitoring of the pose of the marker (and thus, real-time validation of the accuracy of the first imaging device). For example, steps 204-216 may be repeated continuously throughout a surgical operation to validate the pose of the marker. It will also be appreciated steps 204-216 may be repeated upon input received from a user. In other examples, steps 204-228 may be repeated continuously until step 232 (e.g., generating a notification) is triggered, at which point a user may take steps to adjust the first imaging device to improve the accuracy or fix any errors of the first imaging device.

The present disclosure encompasses embodiments of the method 200 that comprise more or fewer steps than those described above, and/or one or more steps that are different than the steps described above.

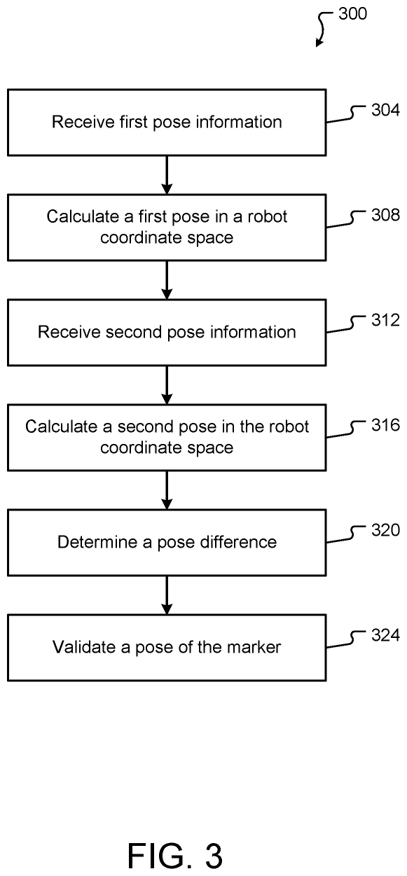

FIG. 3 depicts a method 300 that may be used, for example, validating a pose of a marker.

The method 300 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 300. The at least one processor may perform the method 300 by executing elements stored in a memory such as the memory 106. The elements stored in memory and executed by the processor may cause the processor to execute one or more steps of a function as shown in method 300. One or more portions of a method 300 may be performed by the processor executing any of the contents of memory, such as an image processing 120, validation 122, and/or monitoring 124.

The method 300 comprises receiving first pose information (step 304). The step 304 may be the same as or similar to step 204 of the method 200 described above.

The method 300 also comprises calculating a first pose in a robot coordinate space (step 308). Calculating the first pose may be based on the first pose information received in, for example, step 304 and may be further based on first pose information of a robot reference point. In other words, the first pose may be calculated relative to the robot reference point. The robot reference point may comprise, for example, a robot reference frame and/or a robot landmark. The landmark may be, for example, an arm guide. The first pose information of the robot reference point may be received from a first imaging device such as the imaging device 128 of a navigation system such as the navigation system 118.

The method 300 also comprises receiving second pose information (step 312). The step 312 may be the same as or similar to step 208 of the method 200 described above.

The method 300 also comprises calculating a second pose in the robot coordinate space (step 316). Calculating the second pose may be based on the second pose information received in, for example, step 312 and may be further based on second pose information of the robot reference point. In other words, the second pose may be calculated relative to the robot reference point. The second pose information of the robot reference point may be received from a second imaging device such as the imaging device 112.

The method 300 also comprises determining a pose difference (step 320). The step 320 may be the same as or similar to step 212 of the method 200 described above.

The method 300 also comprises validating a pose of the marker (step 324). The step 324 may be the same as or similar to step 216 of the method 200 described above.

It will be appreciated that the method 300 may include any step of the method 200 described above. For example, the method 300 may include any of steps 220-232 (e.g., calculating and displaying an accuracy index, monitoring a pose difference, and/or automatically generating a notification).

The present disclosure encompasses embodiments of the method 300 that comprise more or fewer steps than those described above, and/or one or more steps that are different than the steps described above.

As noted above, the present disclosure encompasses methods with fewer than all of the steps identified in FIGS. 2 and 3 (and the corresponding description of the methods 200 and 300), as well as methods that include additional steps beyond those identified in FIGS. 2 and 3 (and the corresponding description of the methods 200 and 300 The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system for validating a pose of a marker comprising:
a marker;
a processor; and
a memory storing data for processing by the processor, the data, when processed, causes the processor to:
receive first pose information of the marker;
receive second pose information of the marker;
determine a pose difference between the first pose information and the second pose information; and
validate a pose of the marker in response to determining that the pose difference is less than a pose threshold.

2. The system of claim 1, further comprising a first imaging device and a second imaging device, wherein the first pose information is received from the first imaging device and the second pose information is received from the second imaging device.

3. The system of claim 2, wherein the first imaging device comprises a navigation camera and the second imaging device comprises a three-dimensional camera.

4. The system of claim 2, wherein the second imaging device is positioned on a robot and the marker is positioned on a patient.

5. The system of claim 2, wherein the second imaging device is positioned external to a robot and the marker is positioned on the robot.

6. The system of claim 1, wherein the memory stores further data for processing by the processor that, when processed, causes the processor to:
calculate an accuracy index based on the pose difference; and
display the accuracy index.

7. The system of claim 1, wherein the memory stores further data for processing by the processor that, when processed, causes the processor to:
monitor the pose difference; and
automatically generate a notification when the pose difference exceeds the pose threshold.

8. A system for validating a pose of the marker comprising:
a navigation system comprising a first imaging device;
a second imaging device positioned on a robot;
a processor; and
a memory storing data for processing by the processor, the data, when processed, causes the processor to:
receive first pose information from the first imaging device;
receive second pose information from the second imaging device;
determine a pose difference between the first pose information and the second pose information; and
validate a pose of the marker in response to determining that the pose difference is less than a pose threshold.

9. The system of claim 8, further comprising a marker.

10. The system of claim 9, wherein the marker is positioned on an instrument.

11. The system of claim 9, wherein the marker comprises at least one light emitting diode.

12. The system of claim 10, wherein the instrument comprises a bone mount.

13. The system of claim 10, wherein the first pose information corresponds to a pose of the marker and the second pose information corresponds to a pose of the instrument.

14. The system of claim 8, wherein the first imaging device is a navigation camera and the second imaging device is a three-dimensional camera.

15. A system for validating a pose of the marker comprising:
a marker positioned on a robot;
a navigation system comprising a first imaging device;
a second imaging device positioned external to the robot;
a processor; and
a memory storing data for processing by the processor, the data, when processed, causes the processor to:
receive a first pose information of the marker from the first imaging device;
calculate a first pose of the marker in a robot coordinate space based on the first pose information;
receive a second pose information of the marker from the second imaging device;
calculate a second pose of the marker in the robot coordinate space based on the second pose information;
determine a pose difference between the first pose and the second pose; and
validate a pose of the marker in response to determining that the pose difference is less than a pose threshold.

16. The system of claim 15, wherein calculating the first pose of the marker in the robot coordinate space is further based on first pose information of a robot reference point and calculating the second pose of the marker in the robot coordinate space is further based on second pose information of the robot reference point.

17. The system of claim 15, wherein the robot reference point comprises at least one of a robot reference frame or a robot landmark.

18. The system of claim 15, wherein the first imaging device is a navigation camera and the second imaging device is a three-dimensional camera.

19. The system of claim 15, wherein the marker comprises at least one of a light emitting diode, an infrared light emitting diode, or a reflective sphere.

20. The system of claim 15, wherein the memory stores further data for processing by the processor that, when processed, causes the processor to:
monitor the pose difference; and
automatically generate a notification when the pose difference exceeds the pose threshold.
